(19) United States
(12) Patent Application Publication
(10) Pub. No.: US 2008/0160577 A1
(43) Pub. Date: Jul. 3, 2008

(54) Optimization of Heterologous Polypeptide Expression

(75) Inventors: Philip C. Dell'Orco, King of Prussia, PA (US); Linden Gledhill, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 11/815,487

(22) PCT Filed: Feb. 3, 2006
(86) PCT No.: PCT/US06/03810

§ 371 Date: Aug. 3, 2007

Related U.S. Application Data
(60) Provisional application No. 60/650,348, filed on Feb. 4, 2005.

(51) Int. Cl. C12P 21/00

(52) U.S. Cl. 435/70.3; 435/70.1

(57) ABSTRACT

This invention relates to methods for controlling deamidation of at least one type of heterologously expressed polypeptide in cell culture.

### BACKGROUND

During the growth of cell culture and heterologous expression of polypeptides, several modifications of expressed polypeptide can occur that effect polypeptide function and/or structure. For instance, some modifications include methionine oxidation, glycosylation, gluconoylation, mutations in polypeptide chain sequences, N-terminal glutamine cyclization and deamidation, and asparagine deamidation. Many of these modifications occur spontaneously during cell culture and polypeptide expression. After cell harvest, modified and unmodified polypeptides may be separated, adding to the cost of and reducing the efficiency of production.

Thus, methods for controlling the incidence and/or extent of deamidation of expressed or overexpressed heterologous polypeptides in cell culture is greatly needed.

### SUMMARY OF THE INVENTION

In one aspect of the present invention, methods are provided for controlling deamidation of at least one type of heterologously expressed polypeptide in a cell culture comprising the steps of detecting a total amount of at least one type of heterologously expressed polypeptide in said culture; detecting an amount of deamidated said at least one type of heterologously expressed polypeptide in said cell culture; and harvesting cells at a desired ratio of deamidated to total at least one type of heterologously expressed polypeptide.

In another aspect of the present invention, methods are provided for controlling deamidation of at least one type of heterologously expressed polypeptide in a cell culture comprising the steps of detecting a total amount of at least one type of heterologously expressed polypeptide in said culture; detecting an amount of amidated said at least one type of heterologously expressed polypeptide in said cell culture; and harvesting cells at a desired ratio of amidated to total at least one type of heterologously expressed polypeptide.

In another aspect of the present invention, methods are provided for controlling deamidation of at least one type of heterologously expressed polypeptide in a cell culture comprising the steps of detecting a ratio of amidated and deamidated at least one type of heterologously expressed polypeptide; and harvesting cells at a desired ratio of amidated to deamidated at least one type of heterologously expressed polypeptide.

### BRIEF DESCRIPTION OF THE DRAWINGS

FIG. 1. Data Fits to Non-Cellular System, Time Series at 33.8° C.

FIG. 2. Data Fits to Cellular System, Production Batch-1.

FIG. 3. Data Fits to Cellular System, Production Batch-2.

FIG. 4: Asparagine Deamidation Formation of Asp & pGlu in Mab2 as measured by HPLC.

### DETAILED DESCRIPTION OF THE INVENTION

### Glossary

“Host cell(s)” is a cell, including but not limited to a mammalian cell, insect cell, bacterial cell or cell of a microorganism, that has been introduced (e.g., transformed, infected or transfected) or is capable of introduction (e.g., transformation, infection or transfection) by an isolated and/or heterologous polynucleotide sequence.

“Transformed” as known in the art, is a modification of an organism's genome or episome via the introduction of isolated and/or heterologous DNA, RNA, or DNA-RNA hybrid, or to any other stable introduction of such DNA or RNA.

“Transfected” as known in the art, is the introduction of isolated and/or heterologous DNA, RNA, or a DNA-RNA hybrid, into a host cell or microorganism, including but not limited to recombinant DNA or RNA

“Identity,” means, for polynucleotides and polypeptides, as the case may be, the comparison calculated using an algorithm provided in (1) and (2) below.

(1) Identity for polynucleotides is calculated by multiplying the total number of nucleotides in a given sequence by the integer defining the percent identity divided by 100 and then subtracting that product from said total number of nucleotides in said sequence, or:

nₙ≦xₙ−(xₙ•y),

wherein nₙ is the number of nucleotide alterations, xₙ is the total number of nucleotides in a given sequence, y is 0.95 for 95%, 0.97 for 97% or 1.00 for 100%, and • is the symbol for the multiplication operator, and wherein any non-integer product of xₙ and y is rounded down to the nearest integer prior to subtracting it from xₙ. Alterations of a polynucleotide sequence encoding a polypeptide may create nonsense, missense or frameshift mutations in this coding sequence and thereby alter the polypeptide encoded by the polynucleotide following such alterations.

(2) Identity for polypeptides is calculated by multiplying the total number of amino acids by the integer defining the percent identity divided by 100 and then subtracting that product from said total number of amino acids, or:

nₐ≦xₐ−(xₐ•y),

wherein nₐ is the number of amino acid alterations, xₐ is the total number of amino acids in the sequence, y is 0.95 for 95%, 0.97 for 97% or 1.00 for 100%, and • is the symbol for the multiplication operator, and wherein any non-integer product of xₐ and y is rounded down to the nearest integer prior to subtracting it from xₐ.

“Heterologous(ly)” means (a) obtained from an organism through isolation and introduced into another organism, as, for example, via genetic manipulation or polynucleotide transfer, and/or (b) obtained from an organism through means other than those that exist in nature, and introduced into another organism, as for example, through cell fusion, induced mating, or transgenic manipulation. A heterologous material may, for example, be obtained from the same species or type, or a different species or type than that of the organism or cell into which it is introduced.

“Isolated” means altered “by the hand of man” from its natural state, has been changed or removed from its original environment, or both. For example, a polynucleotide or a polypeptide naturally present in a living organism is not “isolated,” but the same polynucleotide or polypeptide separated from the coexisting materials of its natural state is “isolated”, including but not limited to when such polynucleotide or polypeptide is introduced back into a cell, even if the cell is of the same species or type as that from which the polynucleotide or polypeptide was separated.

“Polynucleotide(s)” generally refers to any polyribonucleotide or polydeoxyribonucleotide, that may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that comprise one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may comprise amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may comprise many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, PROTEINS—STRUCTURE AND MOLECULAR PROPERTIES, 2nd Ed., T. E. Creighton, W.H. Freeman and Company, New York (1993) and Wold, F., Posttranslational Protein Modifications Perspectives and Prospects, pgs. 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

“Recombinant expression system(s)” refers to expression systems or portions thereof or polynucleotides of the invention introduced (e.g, transfected, infected, or transformed) into a host cell or host cell lysate for the production of the polynucleotides and polypeptides of the invention.

“Variant(s)” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusion proteins and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. The present invention also includes include variants of each of the polypeptides of the invention, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

“Microorganism(s)” means a (1) prokaryote, including but not limited to, (a) Bacteria(l)(um), meaning a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsia and Chlamydia trachomitis, (b) an archaeon, including but not limited to Archaebacter, and (2) a unicellular or filamentous eukaryote, including but not limited to, a protozoan, a fungus, a member of the genus Saccharomyces, Kluveromyces, or Candida, and a member of the species Saccharomyces ceriviseae, Kluveromyces lactis, or Candida albicans.

As used herein “one type of heterologously expressed polypeptide” means all variants of a heterologously expressed polypeptide in a host cell, including all modified and unmodified heterologously expressed polypeptide.

As used herein “modified heterologously expressed polypeptide” means any heterologously expressed polypeptide or variant thereof wherein at least one amino acid of said polypeptide comprises a chemical modification. Chemical modifications may include, but are not limited to, methionine oxidation, glycosylation, gluconoylation, N-terminal glutamine cyclization and deamidation, and asparagine deamidation.

As used herein, “gluconoylation” refers to attachment of a gluconic acid derivative to a protein. Gluconoylation may include, but is not limited to, a 6-phosphogluconolactone (6-PGL) adduct formation, acetylation, formylation, deformylation, gluconolactonation, or gluconic acid derivatization.

As used herein, “titer yield” refers to the concentration of a product (e.g., heterologously expressed polypeptide) in solution (e.g., culture broth or cell-lysis mixture or buffer) and may be expressed as mg/L or g/L. An increase in titer yield may refer to an absolute or relative increase in the concentration of a product produced under two defined set of conditions.

As used herein “harvesting” cells refers to collection of cells from cell culture. Cells may be concentrated during harvest to separate them from culture broth, for instance by centrifugation or filtration. Harvesting cells may further comprise the step of lysing the cells to obtain intracellular material, such as, but not limited to polypeptides and polynucleotides. It should be understood by the skilled artisan that certain cellular material, including but not limited to, heterologously expressed polypeptide, may by released from cells during culture. Thus, a product (e.g., a heterologously expressed polypeptide) of interest may remain in culture broth after cells are harvested.

As used herein “controlling” deamidation of a heterologously expressed polypeptide in a cell culture means modulating cell culture growth conditions, such as, but not limited to, culture medium, pH, temperature, and growth time until cell harvest, such that the amount of deamidated heterologous polypeptide obtained from the cell culture comprises a desired percentage of the entire amount of heterologous polypeptide produced in said cell culture.

As used herein “minimum acceptable concentration” means a concentration of at least one type of heterologously expressed polypeptide in culture medium at which concentration, cells may be harvested and the amount of recovered heterologously expressed polypeptide from said culture is a desired amount, such as, for example, an amount predicted to be enough to produce purified and useable heterologously expressed polypeptide. A minimum acceptable concentration may be determined by such factors including, but not limited to, the cost of cell culture and/or the predicted rate of heterologously expressed polypeptide modification after expression. Examples of minimum acceptable concentrations of at least one type of heterologously expressed polypeptide may be in the range of, but not limited to, 25.0 mg/L to 1500.0 mg/L.

As used herein “an acceptable limit of modification” means a concentration of one type of modified heterologously expressed polypeptide that may be removed from total one type of heterologously expressed polypeptide such that a desired quantity of heterologously expressed polypeptide remains that does not comprise said modification. An acceptable limit of modification for one type of heterologously expressed polypeptide may be determined, for example, as a percentage of total heterologously expressed polypeptide or as an independent concentration of a modified polypeptide. An acceptable limit of modification for one type of heterologously expressed polypeptide may range from about 0% to about 90%, or from about 0% to about 50%, or from about 0% to about 15%, or from about 0% to about 10% of total heterologous polypeptide.

As used herein “an acceptable limit of amidated polypeptide” means a concentration of one type of amidated heterologously expressed polypeptide remaining after deamidated heterologously expressed polypeptide is removed from total type of heterologously expressed polypeptide. An acceptable limit of amidated polypeptide may be determined, for example, as a percentage of total heterologously expressed polypeptide or as an independent concentration of amidated heterologously expressed polypeptide. An acceptable limit of an amidated polypeptide may range from about 100% to about 90%, or from about 100% to about 50%, or from about 100% to about 15%, or from about 100% to about 10% of total heterologous polypeptide.

Several schemes are known or proposed for deamidation of polypeptides. For instance, N-terminal glutamine cyclization and deamidation to form pyro-glutamate can occur via the following reaction:

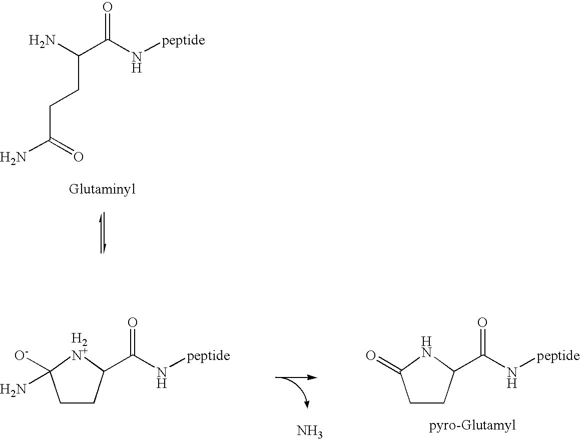

In addition, deamidation of asparagine may occur via the following reaction:

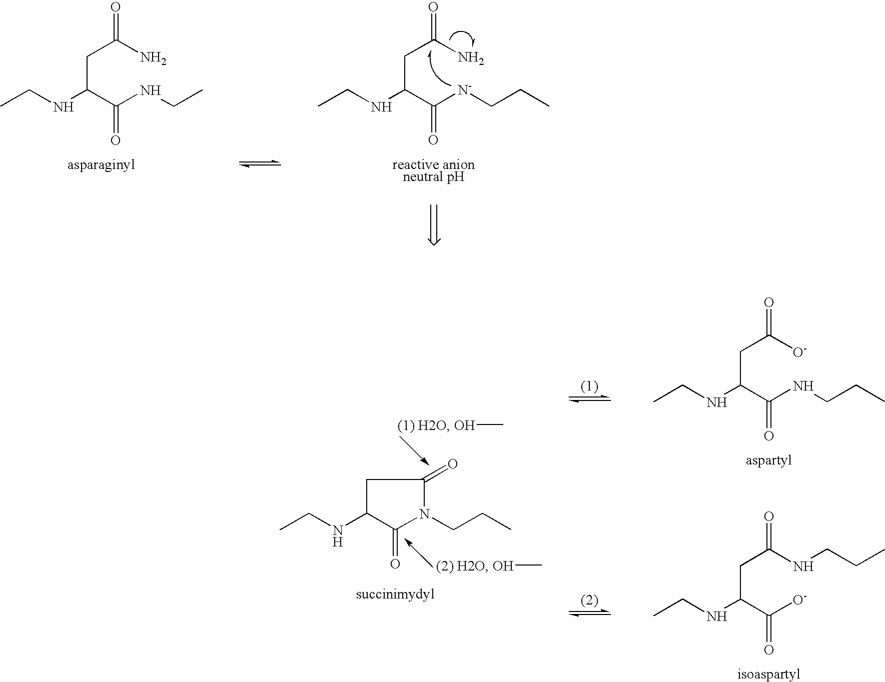

The reaction is important because deamidated products may possess altered structural properties, reduced potency, reduced biological activity, reduced efficacy or allergic and/or immunogenic properties, or other undesirable property.

In one aspect of the present invention, methods are provided for controlling deamidation of at least one type of heterologously expressed polypeptide in a cell culture comprising the steps of detecting a total amount of at least one type of heterologously expressed polypeptide in said culture; detecting an amount of deamidated said at least one type of heterologously expressed polypeptide in said cell culture; and harvesting cells at a desired ratio of deamidated to total at least one type of heterologously expressed polypeptide. A polypeptide may be deamidated at one or more asparagine residues within the amino acid sequence of the polypeptide. Polypeptide deamidation can be measured by several methods, including but not limited to, separations based on charge such as ion exchange or HPLC, isoelectric focusing, capillary electrophoresis, native gel electrophoresis; reversed-phase, hydrophobic interaction, or affinity chromatography; mass spectrometry; or enzymatically using protein L-isoaspartyl methyltransferase.

In another aspect, methods are provided wherein cells are harvested when the total amount of at least one type of heterologously expressed polypeptide reaches a minimum acceptable concentration and the amount of deamidated at least one type of heterologously expressed polypeptide remains equal to or below an acceptable limit of modification. In another aspect, each detecting step comprises using HPLC. In another aspect, at least one detecting step comprises using ion exchange HPLC. Methods are also provided further comprising purifying the total at least one type of said heterologously expressed polypeptide by protein A affinity chromatography. In another aspect, at least one type of said heterologously expressed polypeptide is an antibody. Cell culture may comprise Chinese Hamster Ovary cells. Methods are also provided comprising determining the titer of at least one type of said heterologously expressed polypeptide. In addition, methods are provided for controlling deamidation of at least one type of heterologously expressed polypeptide comprising determining production rates of deamidated at least one type of said heterologously expressed polypeptide.

In another aspect of the present invention, methods are provided for controlling deamidation of at least one type of heterologously expressed polypeptide in a cell culture comprising the steps of detecting a total amount of at least one type of heterologously expressed polypeptide in said culture; detecting an amount of amidated said at least one type of heterologously expressed polypeptide in said cell culture; and harvesting cells at a desired ratio of amidated to total at least one type of heterologously expressed polypeptide. Cells may be harvested when the total amount of at least one type of heterologously expressed polypeptide reaches a minimum acceptable concentration and the amount of amidated at least one type of heterologously expressed polypeptide remains above an acceptable limit of amidated polypeptide. Each detecting step may comprise using HPLC, which may be ion exchange HPLC. Methods are also provided further comprising purifying the total at least one type of said heterologously expressed polypeptide by protein A affinity chromatography. In one aspect, the heterologously expressed polypeptide is an antibody. In another aspect, the cell culture comprises Chinese Hamster Ovary cells. In yet another aspect methods are provided further comprising determining the titer of at least one type of said heterologously expressed polypeptide. Production rates of amidated at least one type of said heterologously expressed polypeptide may also be measured.

In another aspect of the present invention, methods are provided for controlling deamidation of at least one type of heterologously expressed polypeptide in a cell culture comprising the steps of detecting a ratio of amidated and deamidated at least one type of heterologously expressed polypeptide; and harvesting cells at a desired ratio of amidated to deamidated at least one type of heterologously expressed polypeptide. In another aspect, the amount of deamidated said at least one type of heterologously expressed polypeptide is less than the amount of amidated at least one type of heterologously expressed polypeptide in said cell culture when cells are harvested. The ratio of deamidated to amidated heterologously expressed polypeptide may be about 1:9 or about 10% of the total amount of one type of heterologously expressed polypeptide made up by deamidated one type of heterologously expressed polypeptide. Other ratios include, but are not limited to, about 1.5:8.5, about 2:8, about 3:7 or about 4:6. In another aspect, the detecting step comprises using ion exchange HPLC. The heterologously expressed polypeptide is an antibody and cell culture may comprise Chinese Hamster Ovary cells. The methods may also comprises measuring titer of at least one type of said heterologously expressed polypeptide and/or production rates of amidated and deamidated at least one type of said heterologously expressed polypeptide.

The following examples illustrate various aspects of this invention. These examples do not limit the scope of this invention which is defined by the appended claims

### EXAMPLES

### Example 1

### Kinetic Modeling of Data

### Reaction Sequence for Deamidation

A model proposed to describe polypeptide deamidation, using monoclonal antibody (mAB) production in Chinese Hamster Ovary (CHO) cells is provided as an example. The invention herein is not meant to be limited in any way by this model, or any other model or theory disclosed herein. The exemplified monoclonal antibody had a known number of potential deamidation sites such that the reaction products included a singly deamidated product (herein, “deAmidA”), and a doubly deamidated product (herein “deAmidB”). Because of the limited number of sites, use of an equilibrium model via an imide intermediate was not believed to be practical; because of the apparent absence of isoAsp and cyclic imide species, the reaction simply reduced to a pseudo first order model in mAB. Thus, the following reaction sequence was targeted for modeling:

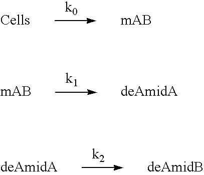

### Reaction Scheme for mAB Production and Deamidation

In the reaction sequence above, mAB is produced by a zero-th order process, dependent only on the number of cells present. The deamidation products are first order in substrate. The implication of the reaction sequence above is the differential equation set shown in Scheme 1, below. The model allows prediction of both singly and doubly deamidated species concentrations. In the differential equation set, singly deamidated product is denoted by deAmidA, while doubly deamidated product is indicated by deAmidB. NCell is the cell titer, and Viability is the percent of cells that are active. Brackets indicate molar species concentrations, and k's represent rate constants.

Scheme 1. Differential Equation Scheme for mAB Production and Deamidation:

$$\frac{d [ mAB ]}{d t} = {N}_{cell} * Viability * {k}_{0} - 2 {k}_{1} * [ mAB ]$$

$$\frac{d [ deAmidA ]}{d t} = 2 {k}_{1} [ mAB ] - {k}_{2} [ deAmidA ]$$

$$\frac{d [ deAmidB ]}{d t} = {k}_{2} [ deAmidA ]$$

Values of k₀, k₁, and k₂ were estimated using the HiQ programming environment (National Instruments Corp., Austin, Tex.). A conjugate gradient optimizer was used to select values of k₀, k₁, and k₂, such that the objective function shown by Equation 1 was minimized. Other known methods may be used to select such values. In Equation 1, shown below, n indicates the time series data points. The factor of 10 multiplier on the doubly deamidated product residual is done to roughly equalize its value with other residuals, thus equalizing its importance in the parameter estimation routine.

$$f = {\sum }_{all n} \{ \begin{matrix}{( {[ mAB ]}_{n , act} - {[ mAB ]}_{n , pred} )}^{2} + \\ {( {[ deAmidA ]}_{n , act} - {[ deAmidA ]}_{n , pred} )}^{2} + \\ ( {[ deAmidB ]}_{n , act} - {[ deAmidB ]}_{n , pred} ) * 10\end{matrix} \} \qquad ( 1 )$$

Data was converted by multiplying peak area ratios by titer values for each component at individual time points. The value of Ncell was continuously updated during the solution to the differential equation by interpolation of actual cell density and viability data. Note that k₀ is analogous to the specific productivity of the culture, which is a measure of mAB accumulation in culture normalized to the cell density in culture.

### Example 2

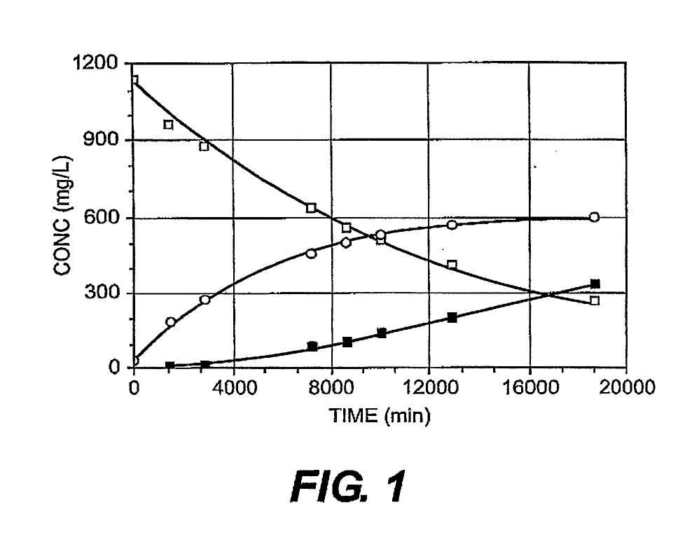

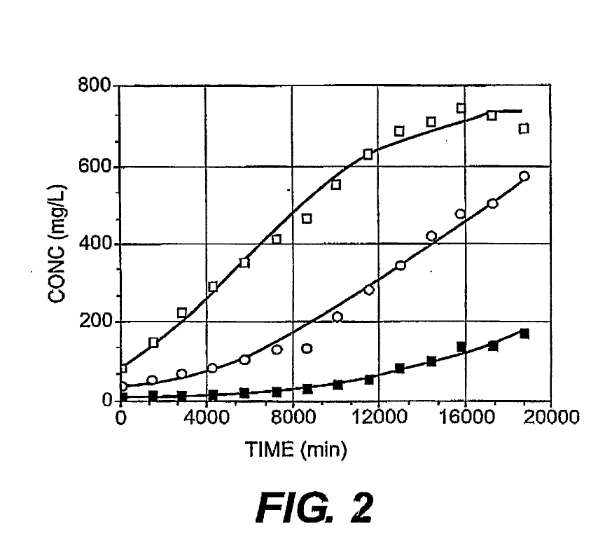

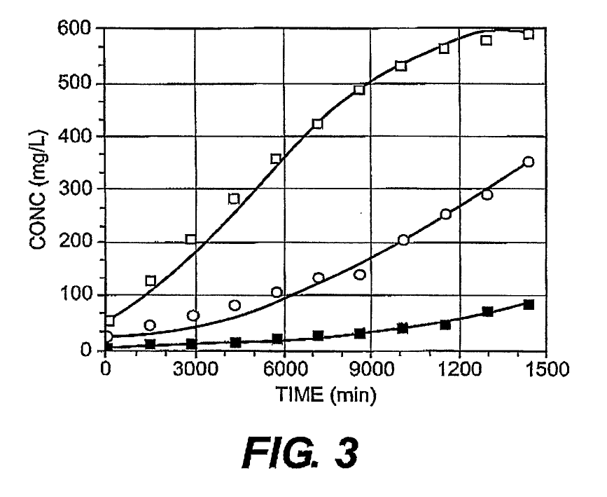

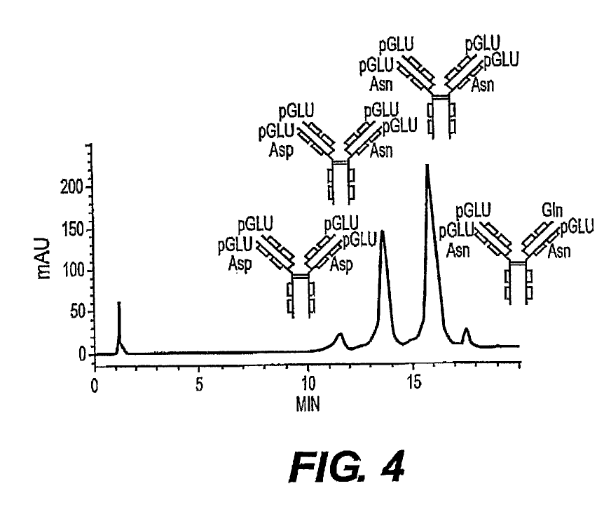

Mab production was measured in cell culture using Chinese Hamster Ovary cells using an HPLC method that quantified the total amount of Mab accumulated. Small aliquots of the culture were harvested and purified using small columns (1-4 milliters) packed with Protein A affinity chromatography media. The purified Mab was subjected to ion exchange HPLC, and the various deamidated species were separated and quantified. An example of deamidated and amidated products as observed by this ion exchange HPLC method is presented in FIG. 4. Using this data, kinetic parameters were determined both in the presence and absence of Chinese Hamster Ovary cells. Table 1 displays the rate constants determined for each experiment. Given the assumptions used in preparing the model, the values of k₁ and k₂ are essentially the same for the cell-free system as for the actual bioreactor system. The kinetic model used is shown in Schemes 1, above. Data fits are shown in FIGS. 1-3. The fits were all high quality. Substituting any k₁/k₂ values into simulation of another data set provided a reasonable fit to the other data set. Thus, there was no appreciable difference between the cell-free and actual systems, indicating that the presence of cells does not catalyze deamidation. These data also indicated that the cells do not express deamidated products, but that the mAB deamidated extracellularly and spontaneously. There was little difference in values of k₁ and k₂ for the first and second deamidations.

TABLE 1
Rate Constants from Preliminary Kinetic
Analysis of mAB Deamidation

| | kₒ (mg/(L * min)) | k₁ (min⁻¹) | k₂ (min⁻¹) |
|---|---|---|---|
| Time Course at 33.8° C. | NA | 4.05⁻⁻⁰⁵ | 3.95⁻⁻⁰⁵ |
| Production Batch-1 | 0.022 | 3.82⁻⁰⁵ | 3.72⁻⁰⁵ |
| Production Batch-2 | 0.011 | 4.44⁻⁻⁰⁵ | 4.72⁻⁰⁵ |

Using the kinetic model of Scheme 1 described in Example 1, a harvest window (e.g., total amount at least one type of heterologously expressed polypeptide reaches a minimum acceptable concentration and amount of deamidated at least one type of heterologously expressed polypeptide remains equal to or below an acceptable limit of modification) at was determined based on Mab accumulation and the accumulation of deamidated species. Cells were harvested and Mab was recovered during the determined harvest window, optimizing the production of amidated (i.e., not deamidated) Mab in balance with the accumulation of deamidated Mab species. The approach presented herein may be applied to polypeptides capable of undergoing asparagine deamidations, including monoclonal antibodies, among others. Using this model, the nature, the potency and yield of a batch can be selected for certain desired optima if a therapeutic potency of a heterologously expressed polypeptide is known. The model can be used to estimate, project or predict the accumulation of Mab species during batch progression, and the model can account for batch to batch variability in cell growth.

Any patent application to which this application claims priority is also incorporated by reference herein in its entirety as being fully set forth herein.

1. A method of controlling deamidation of at least one type of heterologously expressed polypeptide in a cell culture comprising the steps of detecting a total amount of at least one type of heterologously expressed polypeptide in said culture; detecting an amount of deamidated said at least one type of heterologously expressed polypeptide in said cell culture; and harvesting cells at a desired ratio of deamidated to total at least one type of heterologously expressed polypeptide.

2. The method of claim 1, wherein cells are harvested when the total amount of at least one type of heterologously expressed polypeptide reaches a minimum acceptable concentration and the amount of deamidated at least one type of heterologously expressed polypeptide remains equal to or below an acceptable limit of modification.

3. The method of claim 1, wherein each detecting step comprises using HPLC.

4. The method of claim 3, wherein at least one detecting step comprises using ion exchange HPLC.

5. The method of claim 1, further comprising purifying the total at least one type of said heterologously expressed polypeptide by protein A affinity chromatography.

6. The method of claim 1, wherein at least one type of said heterologously expressed polypeptide is an antibody.

7. The method of claim 1, wherein the cell culture comprises Chinese Hamster Ovary cells.

8. The method of claim 1, further comprising determining the titer of at least one type of said heterologously expressed polypeptide.

9. The method of claim 1, further comprising determining production rates of deamidated at least one type of said heterologously expressed polypeptide.

10. A method of controlling deamidation of at least one type of heterologously expressed polypeptide in a cell culture comprising the steps of detecting a total amount of at least one type of heterologously expressed polypeptide in said culture; detecting an amount of amidated said at least one type of heterologously expressed polypeptide in said cell culture; and harvesting cells at a desired ratio of amidated to total at least one type of heterologously expressed polypeptide.

11. The method of claim 10, wherein cells are harvested when the total amount of at least one type of heterologously expressed polypeptide reaches a minimum acceptable concentration and the amount of amidated at least one type of heterologously expressed polypeptide remains above an acceptable limit of amidated polypeptide.

12. The method of claim 10, wherein each detecting step comprises using HPLC.

13. The method of claim 12, wherein at least one detecting step comprises using ion exchange HPLC.

14. The method of claim 10, further comprising purifying the total at least one type of said heterologously expressed polypeptide by protein A affinity chromatography.

15. The method of claim 10, wherein at least one type of said heterologously expressed polypeptide is an antibody.

16. The method of claim 10, wherein the cell culture comprises Chinese Hamster Ovary cells.

17. The method of claim 10, further comprising determining the titer of at least one type of said heterologously expressed polypeptide.

18. The method of claim 10, further comprising determining production rates of amidated at least one type of said heterologously expressed polypeptide.

19. A method of controlling deamidation of at least one type of heterologously expressed polypeptide in a cell culture comprising the steps of detecting a ratio of amidated and deamidated at least one type of heterologously expressed polypeptide; and harvesting cells at a desired ratio of amidated to deamidated at least one type of heterologously expressed polypeptide.

20. The method of claim 19, wherein the amount of deamidated said at least one type of heterologously expressed polypeptide is less than the amount of amidated at least one type of heterologously expressed polypeptide in said cell culture when cells are harvested.

21. The method of claim 19, wherein the detecting step comprises using ion exchange HPLC.

22. The method of claim 19, wherein at least one type of said heterologously expressed polypeptide is an antibody.

23. The method of claim 19, wherein the cell culture comprises Chinese Hamster Ovary cells.

24. The method of claim 19, further comprising determining the titer of at least one type of said heterologously expressed polypeptide.

25. The method of claim 19, further comprising determining production rates of amidated and deamidated at least one type of said heterologously expressed polypeptide.
